(19) United States
(12) Plant Patent Application Publication
(10) Pub. No.: US 2013/0086720 P1
(43) Pub. Date: Apr. 4, 2013

(54) Phalaenopsis orchid plant named 'Moondust'

(50) ;

(71) Applicant: Floricultura, Heemskerk (NL)

(72) Inventor: René Schoone, Assendelft (NL)

(21) Appl. No.: 13/573,581

(22) Filed: Sep. 26, 2012

Related U.S. Application Data
(60) Provisional application No. 61/540,883, filed on Sep. 29, 2011.

(30) Foreign Application Priority Data
Sep. 30, 2011 (NL) PBR OPS837

(51) Int. Cl. A01H 5/00

(52) U.S. Cl. PLT/311

(57) ABSTRACT

A new and distinct Phalaenopsis plant named ‘Moondust’ particularly characterized by flowers which are light yellow with red/purple spots and stripes: the labellum is purple with some yellow; plants which may be propagated economically and uniformly using tissue culture; plants which produce more than one inflorescence; long and sturdy inflorescences; and relatively short, dark-green foliage.

### LATIN NAME OF THE GENUS AND SPECIES OF THE PLANT CLAIMED

Phalaenopsis hybrida

### VARIETY DENOMINATION

‘Moondust’

### CROSS-REFERENCE TO RELATED APPLICATIONS

This application claims priority to U.S. Provisional Application No. 61/540,883 filed Sep. 29, 2011, and Netherlands Plant Breeders' Rights application no. OPS837 filed Sep. 30, 2011. The disclosures of both prior applications are hereby incorporated by reference.

### BACKGROUND OF THE INVENTION

The present invention relates to a new and distinct cultivar of Phalaenopsis plant, botanically known as Phalaenopsis of the Orchidaceae family, and hereinafter referred to by the cultivar name ‘Moondust’.

Phalaenopsis comprises a genus of about 55 species of herbaceous perennials many of which, or the hybrids thereof, are suitable for cultivation in the home or greenhouse. Phalaenopsis is predominantly epiphytic or rock-dwelling, and is native to tropical Asia, the Malay Archipelago, and Oceania. The species typically has 2-ranked, fleshy, oblong or elliptic leaves affixed to a short central stem (monopodial growth), which vary in size from 5 to 8 inches to over 2 feet. The leaves may be entirely green or mottled with silver grey.

Phalaenopsis orchids, often referred to as ‘Moth Orchids’ in the horticultural trade, are frequently used to furnish cut flowers for the florist trade or sold as flowering potted-plants for home or interiorscape.

Phalaenopsis produces upright or pendent lateral racemes, often with many showy flowers which open in succession beginning with the lowermost. The flowers possess three sepals and three petals; the lateral ones being alike. The lowermost petal, called the labellum, is three-lobed and is often more brightly-colored than the other flower segments. Flower colors include various shades of pink, white, yellow and red-brown.

Phalaenopsis orchids are typically propagated from seeds. Asexual propagation of Phalaenopsis is often done from off-shoots which frequently arise from the lower bracts of the inflorescence. The resulting plants are detached from the mother plant and may be planted in a suitable substrate.

The new Phalaenopsis ‘Moondust’ is a product of a controlled breeding program conducted by the inventors, René Schoone, in Strengweg, Heemskerk, The Netherlands. The objective of the breeding program was to develop a new Phalaenopsis cultivar particularly characterized by its attractive and unique colored flowers, economical propagation via tissue culture, rapid growth, and a plant dimension suitable for packaging and shipping to the market.

The new Phalaenopsis ‘Moondust’ originated from a cross made by the inventor in 1999 in Strengweg, Heemskerk, The Netherlands. The female or seed parent is the Phalaenopsis cultivar designated ‘Fortune Buddha’, unpatented. The male or pollen parent is the Phalaenopsis cultivar designated ‘Taipei Gold’, unpatented. The new Phalaenopsis ‘Moondust’ was discovered and selected by the inventor as a single flowering plant within the progeny of the stated cross in a controlled environment in 2006 in Strengweg, Heemskerk, The Netherlands.

Asexual reproduction of the new Phalaenopsis cultivar by tissue culture was first performed in July, 2007 in Cieweg 13, Heemskerk, The Netherlands, and has demonstrated that the combination of characteristics as herein disclosed for the new cultivar are firmly fixed and retained through successive generations of asexual reproduction. The new cultivar reproduces true to type.

### BRIEF DESCRIPTION OF THE INVENTION

The following traits have been repeatedly observed and are determined to be unique characteristics of ‘Moondust’, which in combination distinguish this Phalaenopsis as a new and distinct cultivar:

1. flowers which are light yellow with red/purple spots and stripes; the labellum is purple with some yellow;

2. plant produces more than one inflorescence;

3. plants may be propagated economically and uniformly using tissue culture;

4. inflorescence is long and sturdy

5. relatively short, dark-green foliage

Presently, there is no commercial cultivar to which ‘Moondust’ can be meaningfully compared.

### BRIEF DESCRIPTION OF THE PHOTOGRAPHS

The accompanying photographs illustrate the overall appearance of the new Phalaenopsis ‘Moondust’ showing the colors as true as is reasonably possible with colored reproductions of this type. Colors in the photographs may differ slightly from the color values cited in the detailed botanical description, which accurately describe the color of ‘Moondust’.

FIG. 1 shows a side view perspective of a typical flowering plant of ‘Moondust’ in a 12 cm pot, at 16 months of age.

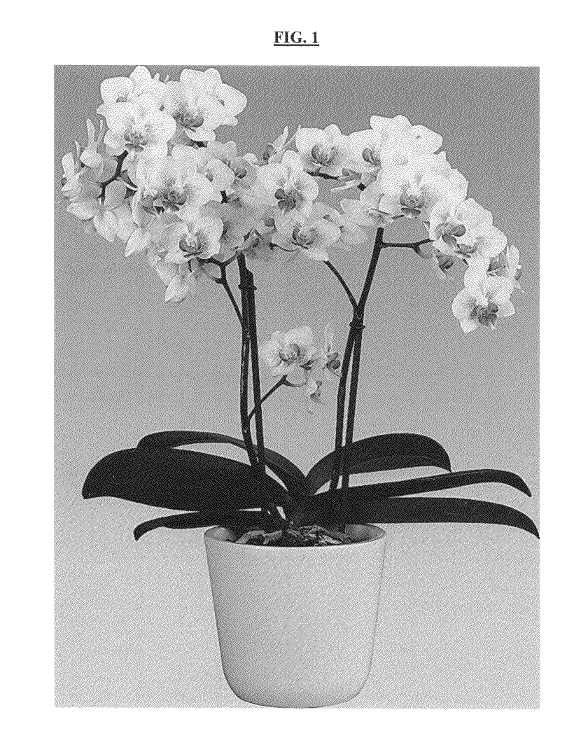

FIG. 2 shows a close-up view of the typical buds and flowers of ‘Moondust’.

FIG. 3 shows a close-up view of the typical leaves of ‘Moondust’.

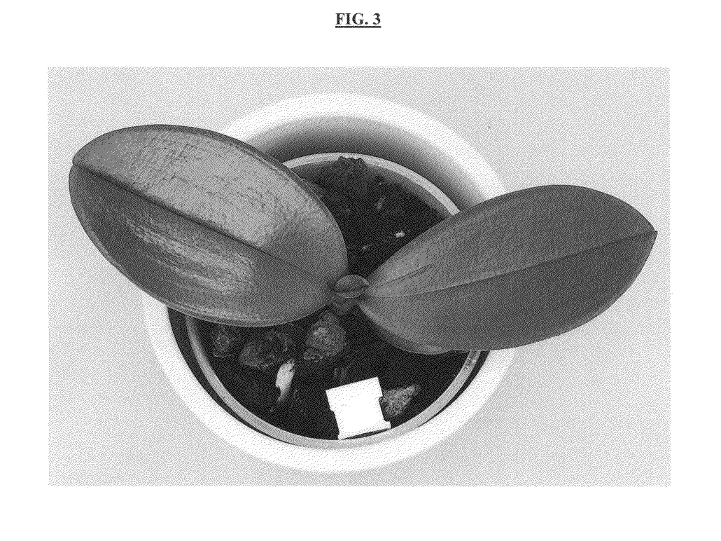

### DETAILED BOTANICAL DESCRIPTION

The new Phalaenopsis cultivar ‘Moondust’ has not been observed under all possible environmental conditions: The phenotype of the new cultivar may vary with variations in environment such as temperature, light intensity, and day length without any change in the genotype of the plant.

The aforementioned photographs, together with the following observations, measurements and values describe plants of ‘Moondust’ as grown in a greenhouse in Strengweg, Heemskerk, The Netherlands, under conditions which closely approximate those generally used in commercial practice. Initially, the ideal temperature to grow plants of ‘Moondust’ is 27° C. during the day and at night. Then, during the flowering phase of ‘Moondust’, the ideal growing temperature is 20-22° C. during the day and 18° C. at night. Light levels for growing ‘Moondust’ are a minimum of 5,000 lux and a maximum of 10,000 lux. A balanced fertilizer with level of 200 ppm N, 87 ppm P, 168 ppm K is applied. Duration of growth of ‘Moondust’ from potting size is between 10 and 14 months.

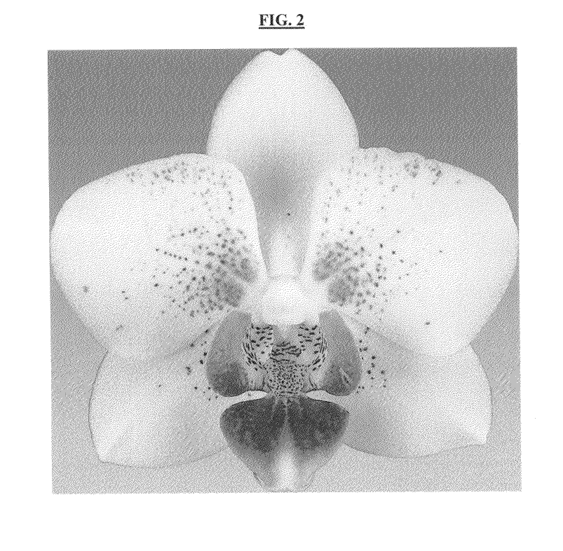

Color references are made to The Royal Horticultural Society Colour Chart (R.H.S.), 2007 edition, except where general colors of ordinary significance are used. Color values were taken under daylight conditions at approximately noon in Zaandammerweg, Assendelft, The Netherlands. The age of the ‘Moondust’ plants described is 12 months after potting.
Classification:
   Botanical.—Phalaenopsis.
Parentage:
   Female or seed parent.—Phalaenopsis cultivar designated ‘Fortune Buddha’, unpatented.
   Male or pollen parent.—Phalaenopsis cultivar designated ‘Taipei Gold’, unpatented.
Propagation:
   Type.—tissue culture.
   Rooting habit and description.—Fleshy; approximately 4 mm-8 mm wide and green/yellow in color (RHS 146C); freely branching. It takes 12 weeks for plants growing in tissue culture to initiate roots.
Plant:
   Size at maturity.—Height: about 40 to 45 cm Spread: about 35 to 43 cm.
   Growth habit.—small; dark-green leaves and relatively normal raceme.
   Vigor.—moderate.
   Crop time.—Following asexual propagation, at about 26 weeks 2 leaves appear; at about 30 weeks 3-4 leaves appear; after a cold treatment of about 4-8 weeks at a temperature of about 19° C. about 2 peduncles with flowers appear.
Foliage:
   Quantity per plant.—About 6 to 8 leaves are produced before flowering.
   Arrangement and attachment.—half up/horizontal and on two sides.
   Overall shape of leaf.—oval, tip is blunt and asymmetric.
   Texture.—smooth and leathery.
   Pubescence.—3 to t4 pairs of leaves.
   Mature leaf length.—about 16 to 21 cm.
   Mature leaf width.—about 9 to 11 cm.
   Mature leaf thickness.—about 2 mm.
   Mature leaf color.—upper surface: RHS 139A; under surface: RHS 137B.
   Venation.—Pattern: parallel Color of midvein: upper surface: RHS 147A; under surface: RHS 139B.
Peduncle:
   Quantity per plant.—about 1 to 2.
   Number of flowers per peduncle.—about 10 to 15.
   Length.—about 36 to 40 cm.
   Diameter.—about 4 to 6 mm.
   Strength.—strong.
   Aspect.—upright.
   Texture.—glabrous and smooth.
   Color.—green (RHS 137B).
   Internode.—Length: about 23 to 30 mm.
Inflorescence description:
   Appearance.—upright to slightly pendant, racemose inflorescence with bilaterally symmetrical flowers that open in succession beginning with the lowermost flower.
   Inflorescence size.—Height (from base to tip): about 15 to 20 mm Diameter (at midpoint): about 10 to 15 mm Shape: egg-shaped Color: yellow/green (RHS 144B).
   Flowering time.—For an untreated plant (flowering plant that has not undergone cold-treatment where the plant grows at a temperature of 18° C. to 19° C. for about 4 to 8 weeks after a period of about 30 weeks at a temperature of 25° C.), 2 racemes appear with about 16 to 20 flower buds and flowers per inflorescence. First flowers can be expected approximately 4 to 6 months after planting a plant with a leaf diameter of 3 to 5 cm. Flowers persistent.
   Flowering longevity.—On the plant: about 4 to 6 months; lastingness of cut flowers: has not been observed.
   Fragrance.—darker/younger flowers no fragrance; lighter/older flowers fragrance.
   Flower.—Rate of opening: Flowers fully opened about 2 to 3 days after tepal separation. Orientation at opening: slanted upward and outward. Shape: see FIG. 2 Size: Height: about 45 to 55 mm Diameter: about 50 to 55 mm Depth of tube: about 8 mm.
   Tepals.—Quantity and arrangement: six tepals that are trimerous, overlapping and arranged in 2 whorls. Petals are more pronounced than sepals.
   Petals.—Arrangement: Inner whorl of tepals comprises 3 petals, 2 lateral petals and labellum. 2 lateral petals: Overall shape: little bit triangular/broadly ovate and weakly cupped Apex: oval Margin: weakly undulate Length: about 22 to 27 mm Width: about 22 to 28 mm Texture: Upper surface: smooth and satiny Under surface: smooth and satiny Color (when fully opened): Main color is yellow (RHS 4D) (The younger/newer flowers are green/yellow (RHS 1 C), mainly at the base some red/purple spots and stripes (RHS N74B). Under surface: older flower is white (RHS 155A), younger flower is green/yellow (RHS 1C). Labellum: Overall shape: 3-lobed with 2 prominent callosities at central junction of the lateral lobes and base of the midlobe. Lateral lobes of labellum fold upward about the column; the midlobe extends forward and is terminated by 2 short filiform appendages at the apex. Lateral lobes of the labellum are ovate in shape while the midlobe is triangular with a bump and a rib on it Margin: entire and weakly undulate Length: about 19 mm Width (not flattened): about 15 mm Texture: Upper & under surface: smooth and satiny Color (when fully opened): Main color lobs is purple (RHS N78B); mid lob has red/purple spots (RHS 60B) and the base, a line in the center and thin edges are yellow (RHS 7A). Chirri is yellow (RHS 5B). Lower edge lateral lobs is red/purple (RHS 60B) and the rest is yellow (RHS 6C) with red/purple spots and stripes (RHS 59A) Chirri: two small points Pestle Length: about 4 mm Width (not flattenend): about 5 mm Height: about 5 mm Color: yellow (RHS 7A) with red/purple spots and stripes (RHS 59A).
   Sepals.—Arrangement: Outer whorl of tepals comprises 3 sepals. Overall shape: elliptical and weakly cupped; v-shaped hole on top of dorsal sepals Length: about 25 to 33 mm Width: about 20 to 22 mm Texture: Upper & under surface: smooth and satiny Color (when fully opened): young flower upper surface: green/yellow (RHS 1 C) with red/purple spots & stripes (lateral sepal RHS 60A, dorsal sepal RHS N74B). Under surface is green/yellow (RHS 1C). Older flower upper surface: yellow (RHS 4D) with red/purple spots & stripes (lateral sepal RHS 60A, dorsal sepal RHS N74B). Under surface is yellow (RHS 4D).
Reproductive organs:
   Arrangement.—The stamens, style and stigmas are fused into a single, short structure called the column, possessing one terminal anther with pollen grains united into a pollinia, which are covered by an anther cap. The stigma is located under the column behind the pollinia. The ovary is inferior with three carpels present. The plant has not produced seed.
   Column.—Length: about 8 mm Diameter: about 5 mm Color: white (RHS NN155C_.
   Pollinia.—Quantity: Two Size: about 1 mm Color: orange (RHS 24A).
   Ovary.—Length: about 4 mm Diameter: about 5 mm Color: white (RHS NN155C).
   Pedicel.—Length: about 23 to 30 mm Diameter: about 3 mm Texture: glabrous and smooth Color: Base is green (RHS 136B), middle is yellow/green (RHS 147C) and close to flower is (RHS 75B).
Disease/pest resistance/susceptibility: No specific resistance or susceptibility observed
Temperature tolerance: Tolerant to a low temperature of about 15° C. and to a high temperature about 30° C.

What is claimed is:

1. A new and distinct Phalaenopsis plant named ‘Moondust’, as illustrated and described herein.
